(19) United States
(12) Patent Application Publication
(10) Pub. No.: US 2014/0171693 A1
(43) Pub. Date: Jun. 19, 2014

(54) Coated Hydrotalcite Catalysts and Processes for Producing Butanol

(71) Applicant: CELANESE INTERNATIONAL CORPORATION, Irving, TX (US)

(72) Inventors: Cheng Zhang, Houston, TX (US); Mason Borlik, Naperville, IL (US); Heiko Weiner, Pasadena, TX (US)

(73) Assignee: CELANESE INTERNATIONAL CORPORATION, Irving, TX (US)

(21) Appl. No.: 13/719,857

(22) Filed: Dec. 19, 2012

(51) Int. Cl. B01J 27/236; C07C 29/32

(52) U.S. Cl. CPC B01J 27/236; C07C 29/32
USPC 568/902.2; 502/176

(57) ABSTRACT

A catalyst composition for converting ethanol to higher alcohols, such as butanol, is disclosed. The catalyst composition comprises metal coated hydrotalcite and method of making same.

### FIELD OF THE INVENTION

The present invention relates generally to a process of making higher molecular weight alcohols from ethanol and, in particular, to a catalytic conversion of ethanol to butanol.

### BACKGROUND OF THE INVENTION

Studies have been done for economically viable processes to produce butanol. Like ethanol, butanol may be a possible solution to dependency on oil as both may be used as a fuel in an internal combustion engine. In fact, due to the longer hydrocarbon chain and non-polar characteristics, butanol may be a better fuel option than ethanol because butanol is more similar to gasoline than ethanol. In addition, butanol may be used in the manufacture of pharmaceuticals, polymers, pyroxylin plastics, herbicide esters and butyl xanthate. Butanol may also be used as a solvent for the extraction of essential oils or as an ingredient in perfumes; as an extractant in the manufacture of antibiotics, hormones, and vitamins; as a solvent for paints, coatings, natural resins, gums, synthetic resins, alkaloids, and camphor. Other applications of butanol includes as swelling agent in textiles; as a component of break fluids, cleaning formulations, degreasers, and repellents; and as a component of ore floatation agents and of wood-treating systems.

Butanol is typically produced industrially from petrochemical feedstock propylene in the presence of a rhodium-based homogeneous catalyst. During this process, propylene is hydroformylated to butyraldehyde and butyraldehyde is then hydrogenated to product butanol. However, due to the fluctuating natural gas and crude oil prices the cost of producing butanol using this method also becomes more unpredictable and significant.

It is known that butanol may be prepared by condensation from ethanol over basic catalyst at high temperature using the Guerbet reaction. The reaction mechanism for the conversion of ethanol to butanol via the Guerbet reaction comprises a four-step sequence as shown in reaction scheme 1. In the first step, ethanol is oxidized to intermediate aldehyde and two of the intermediate aldehydes undergo an aldol condensation reaction to form crotonaldehyde, which is reduced to butanol via hydrogenation. See, for example, J. Logsdon in Kirk-othmer Encyclopedia of Chemical Technology, John Wiley and Sons, Inc., New York, 2001; J. Mol. Catal. A: Chem., 2004, 212, p. 65; and J. Org. Chem., 2006, 71, p. 8306.

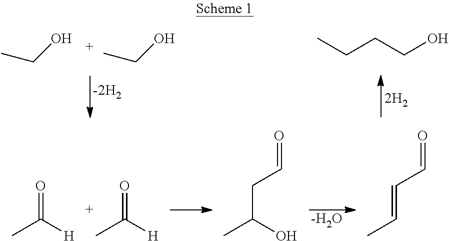

Various catalysts have been studied to improve the conversion and selectivity of ethanol to butanol. For example, M. N. Dvomikoff and M. W. Farrar, J. of Organic Chemistry (1957), 11, 540-542, discloses the use of a MgO—K₂CO₃—CuCrO₂ catalyst system to promote ethanol condensation to higher alcohols, including butanol. U.S. Pat. No. 5,300,695 discloses processes where an L-type zeolite catalyst, such as a potassium L-type zeolite, is used to react with an alcohol having X carbon atoms to produce alcohol with higher molecular weight.

The use of hydroxyapatite Ca₁₀(PO₄)₆(OH)₂, tricalcium phosphate Ca₃(PO₄)₂, calcium monohydrogen phosphate CaHPO₄.(0-2)H₂O, calcium diphosphate Ca₂P₂O₇, octacalcium phosphate Ca₈H₂(PO₄)₆.5H₂O, tetracalcium phosphate Ca₄(PO₄)₂O, or amorphous calcium phosphate Ca₃(PO₄)₂.nH₂O, to convert ethanol to higher molecular weight alcohols are disclosed in WO2006059729.

Hydrotalcites have also been studied as catalysts for making butanol from ethanol. For example, J. I. DiCosimo, et al. discloses the use of Mg_{y}AlOₓ catalysts for alcohol reactions, including ethanol. Journal of Catalysis (1998), 178, 499-510; Journal of Catalysis (2000), 190, 261-275; and Journal of Catalysis (2003) 215, 220-233. U.S. Pat. Nos. 7,705,192 and 7,700,810 disclose the use of partially or fully thermally decomposed hydrotalcites for the conversion of ethanol to butanol. U.S. Pat. No. 7,700,812 discloses the incorporation of the anion of ethylenediaminetetraacetic acid with hydrotalcites for the conversion of ethanol to isobutanol and butanol, respectively. U.S. Pat. No. 7,700,811 discloses hydrotalcite/metal carbonate combinations for the conversion of ethanol to butanol.

Carlini et al., Journal of Molecular Catalysis A: Chemical (2005), 232, 13-20, discloses bifunctional heterogeneous hydrotalcites for converting methanol and n-propanol to isobutyl alcohol.

The references mentioned above are hereby incorporated by reference.

As such, the need remains for improved catalysts for making butanol from ethanol, especially those having improved activity and selectivity to butanol.

### SUMMARY OF THE INVENTION

In a first embodiment, the present invention is directed to a process for producing a catalyst composition for converting alcohols to higher alcohols. The process comprises the steps of coating hydrotalcite with one or more metal precursors to form a metal-containing hydrotalcite and calcining the metal-containing hydrotalcite to form the catalyst composition. In a preferred embodiment, the one or more metal precursors comprises one or more metals selected from the group consisting of magnesium, aluminum, gallium, germanium, tin, lead, copper, and other transition metals. A secondary metal precursor to a secondary metal may also be used. Suitable secondary metals include lithium, sodium, potassium, rubidium, cesium, francium, magnesium, calcium, strontium, or barium.

In a second embodiment, the present invention is directed to a catalyst composition for converting alcohols to higher alcohols produced according to the process comprising the steps of coating hydrotalcite with one or more metal precursors to form a metal-containing hydrotalcite and calcining the metal-containing hydrotalcite to form the catalyst composition. In a preferred embodiment, the catalyst is of the formula: HT-M, wherein HT=Mg₆Al₂CO₃(OH)₁₆.4(H₂O) and wherein M is one or more metals selected from the group consisting of magnesium, aluminum, gallium, germanium, tin, lead, copper, and other transition metals.

In a third embodiment, the present invention is to a process for producing higher alcohols. The process comprises the steps of feeding a gaseous stream comprising ethanol over a catalyst composition in a reactor to form higher alcohols, wherein the catalyst composition comprises a hydrotalcite coated with one or more metals, wherein the one or more metals are selected from the group consisting of magnesium, aluminum, gallium, germanium, tin, lead, copper, and other transition metals.

In a fourth embodiment, the present invention is to a composition for converting alcohols to higher alcohols, the catalyst is of the formula: HT-Mₐ-M′_{b}-M″_{c}-A_{d}, wherein HT is Mg₆Al₂CO₃(OH)₁₆.4(H₂O); M is germanium, tin, palladium, or magnesium; M′ is magnesium, aluminum, or copper; M″ is aluminum or copper, provided that M, M′, and M″ are not the same; A is lithium, sodium, potassium, rubidium, cesium, or francium; a is 0.001 to 1, b is 0 to 2, c is 0 to 2, and d is 0 to 2. In one embodiment the formula may comprise lithium/palladium on HT.

### DETAILED DESCRIPTION OF THE INVENTION

### Introduction

The present invention generally relates to a process for synthesizing a linear multi-carbon alcohol from an alcohol having two or fewer carbons that is useful as a chemical industry raw material and fuel composition or a mixture thereof.

Production of multi-carbon alcohols, like butanol, using most conventional processes has been limited by economic and environmental constraints. One of the best known processes is the Guerbet reaction. Specifically, ethanol may be used as the starting material to product butanol. However, intermediates of the reaction can form competing by-products and may lead to impurities in the butanol product. For example, diethyl ether and ethylene may be formed due to the dehydration of ethanol in the presence of an acidic catalyst. 1-hexanol may also be formed via the addition of aldehyde to butyraldehyde, a crotonaldehyde intermediate. Butyraldehyde may also react with other intermediates to form 2-ethylbutanol and 2-ethylhexanol. A crude mixture of the multi-carbon alcohol and impurities may increase the purification needed to recover butanol.

Catalysts, such as multi-catalyst systems, hydroxyapatite, and phosphate derivatives have been used to optimize the yields and selectivity to butanol. In addition, process conditions for the Guerbet reaction have also been studied to optimize the yields and selectivity to butanol.

The Guerbet reaction converts two moles of ethanol to one mole of butanol through multiple intermediates. The reaction comprising first oxidizing ethanol to form an aldehyde, condensing the aldehydes to 3-hydroxy-butyraldehyde, dehydrating the 3-hydroxy-butyraldehyde to crotonaldehyde, and reducing the crotonaldehyde to butanol.

It has now been discovered that certain catalysts effectively oxidizes ethanol to form an intermediate aldehyde, which forms crotonaldehyde, and reduces crotonaldehyde to butanol. Preferably, these catalysts of the present invention serve as a base to oxidize ethanol and to promote aldol condensation, and also as a hydrogenating site for crotonaldehyde to form butanol. Surprisingly and unexpectedly, the inventors found that a catalyst system of hydrotalcite (HT) coated with one or more metals beneficially results in the improvement of ethanol conversion, and/or butanol selectivity of butanol. For purposes of this application, linear multi-carbon alcohols are preferred and thus butanol refers to n-butanol unless otherwise indicated.

### Catalyst Composition

In one embodiment, the present invention is to a metal coated hydrotalcite (HT). The metal coated HT comprises one or more metals (M) selected from the group consisting of magnesium, aluminum, gallium, germanium, tin, lead, copper, and other transition metals. Suitable transition metals include, but are not limited to, iron, nickel, palladium, and cobalt. The metal may be present as a metallic metal or a metal oxide. The one or more metals are preferably selected from the group consisting of magnesium, aluminum, gallium, germanium, tin, lead, palladium and copper.

In some embodiments, combinations of metals may comprise magnesium and at least one of copper, palladium and aluminum. For example, metal combinations may include magnesium/copper, magnesium/palladium, and magnesium/aluminum/copper. The metal coated hydrotalcite may further comprise one or more secondary metals selected from the group consisting of lithium, sodium, potassium, rubidium, cesium, francium, magnesium, calcium, strontium, and barium. In particular, a metal combination of lithium and transition metal is preferred, such as lithium/palladium.

“Hydrotalcite” (HT) as used in the present application generally refers to a commercially available hydrotalcite such as magnesium aluminum hydroxycarbonate, having a chemical formula: Mg₆Al₂CO₃(OH)₁₆.4H₂O. Synthetic magnesium aluminum hydroxycarbonate may be obtained from Sigma-Aldrich. Of course, synthesized or natural hydrotalcite having a similar chemical formula may also be used to make the catalyst composition.

In one embodiment, the catalyst comprises a metal-containing hydrotalcite (HT). For example, the catalyst comprises from 70 wt. % to 99.9 wt. % hydrotalcite and from 0.1 wt. % to 30 wt. % metal, e.g., comprises from 75 wt. % to 95 wt. % hydrotalcite and 5 wt. % to 25 wt. % metal, or comprises from 80 wt. % to 90 wt. % hydrotalcite and 10 wt. % to 20 wt. % metal. The metal wt. % includes all the metals, including the secondary metals if present.

In one embodiment, the hydrotalcite is coated with one or more metals (M) selected from the group consisting of magnesium, aluminum, germanium, tin, lead, palladium, and copper. The coating may form a multi-layer metal-coated hydrotalcite, and as such, one or more layers of metals may be coated on the hydrotalcite. In one embodiment, the one or more layers of metals are different metals and may be coated on hydrotalcite separately. The secondary metal, if present, may also be coated on the hydrotalcite separately. In other embodiments, different metals may be mixed together prior to coating and coated on hydrotalcite together.

“Coat,” “coated,” or “coating” as used in the present application generally refers to one or more metals distributed on the surface of hydrotalcite. This distribution on the surface forms a metal-coated hydrotalcite complex.

The metal-coated hydrotalcite complex may be represented by the formula: HT-M. The catalyst may comprise hydrotalcite and metals selected from the group consisting of magnesium, aluminum, gallium, germanium, tin, lead, copper, and other transition metals. The metal loadings vary depending on the metal. In one embodiment, the catalyst may comprise hydrotalcite and gallium, germanium, or tin, and the metal loading of HT-M composition may range from 0.1 wt % to 20 wt. %, e.g., from 0.5 wt. % to 18 wt. %, or from 1 wt. % to 15 wt. %. In another embodiment, the catalyst may comprise hydrotalcite and palladium, and the HT-M composition comprises from 0.1 wt % to 10 wt. % palladium, e.g., from 0.5 wt. % to 8 wt. %, or from 1 wt. % to 7 wt. %.

In another embodiment, the catalyst comprises hydrotalcite (HT) with two metals (M and M′) selected from the group consisting of magnesium, aluminum, gallium, germanium, tin, lead, copper, and other transition metals. The two metals coated hydrotalcite complex may be represented by the formula: HT-M-M′. The catalyst may also comprise a secondary metal (A) and the formula may be represented by the formula: HT-M-A. The secondary metal (A) may be selected from the group consisting of lithium, sodium, potassium, rubidium, cesium, francium, magnesium, calcium, strontium, and barium. In one exemplary embodiment, the catalyst may comprise hydrotalcite, magnesium and copper, and the HT-M-M′ composition comprises from 0.1 wt. % to 10 wt. % magnesium, e.g., from 0.5 wt. % to 8 wt. %, or from 1 wt. % to 7 wt. % and from 0.1 wt. % to 10 wt. % copper, e.g., from 0.5 wt. % to 8 wt. %, or from 1 wt. % to 7 wt. %. In another embodiment, the catalyst may comprise hydrotalcite, magnesium and palladium, and the HT-M-M′ composition comprises from 0.1 wt. % to 10 wt. % magnesium, e.g., from 0.5 wt. % to 8 wt. %, or from 1 wt. % to 7 wt. % and from 0.1 wt. % to 10 wt. % palladium, e.g., from 0.5 wt. % to 8 wt. %, or from 1 wt. % to 7 wt. %. In yet another embodiment, the catalyst may comprise palladium-lithium and hydrotalcite, and the HT-M-A composition comprises from 0.01 wt. % to 20 wt. % palladium, e.g., from 0.05 wt. % to 18 wt. %, or from 0.1 wt. % to 16 wt. %, and from 0.1 wt. % to 20 wt. % lithium, e.g., from 0.5 wt. % to 18 wt. %, or from 1 wt. % to 16 wt. %. The catalyst composition with two metals may also comprises from 60 wt. % to 99.89 wt. % hydrotalcite, e.g., 64 wt. % to 99.45 wt. % or from 68 wt. % to 98.9 wt. %.

In one embodiment, the catalyst may comprise hydrotalcite with three metals or more metals, provided that at least one of the metals is selected from the group consisting of magnesium, aluminum, gallium, germanium, tin, lead, copper, and other transition metals. The hydrotalcite with three metals or more complex may be represented by the formula: HT-M-M′-M″. The catalyst may also comprise a secondary metal (A) and the formula may be represented by the formula: HT-M-M′-A or HT-M-M′-M″-A. In one embodiment, the catalyst may comprise hydrotalcite, magnesium, aluminum and copper. In one embodiment, the HT-M-M′-M″ composition comprises from 0.1 wt. % to 10 wt. % magnesium, e.g., from 0.5 wt. % to 8 wt. %, or from 1 wt. % to 7 wt. %; from 0.1 wt. % to 10 wt. % aluminum, e.g., from 0.5 wt. % to 8 wt. %, or from 1 wt. % to 7 wt. %; and from 0.1 wt. % to 10 wt. % copper, e.g., from 0.5 wt. % to 8 wt. %, or from 1 wt. % to 7 wt. %.

In one embodiment, the catalyst corresponds to the formula:

HT-Mₐ-M′_{b}-M″_{c}-A_{d}

wherein a, b, c, and d are the relative molar amounts (relative to 1) of hydrotalcite, first metal (M), second metal (M′) and third metal (M″), respectively in the catalyst. The secondary metal (A) is selected from the group consisting of lithium, sodium, potassium, rubidium, cesium, francium, magnesium, calcium, strontium, and barium. In one embodiment, a is 0.001 to 1, and b, c, and d are each independently 0 to 2. It has now been found that the metal-coated hydrotalcite catalysts surprisingly achieve unexpectedly high ethanol conversion in comparison to other hydrotalcite catalysts. Ethanol conversion of at least 28%, e.g., at least 30%, or at least 40%, may be achieved with the metal-coated hydrotalcite catalyst compositions. Surprisingly and unexpectedly, this increase of ethanol conversion is achieved with improved selectivity to butanol. For example, selectivity to butanol of at least 30%, e.g., at least 40%, or at least 50%. Without being bound by theory, it is postulated that metals contain specific functional properties for the Guerbet reaction. As a result of coating the surface of hydrotalcite, the coating metals of the catalyst composition may drive the Guerbet reaction favorably for both ethanol conversion and selectivity to butanol as compared to homogeneous co-precipitation of metal-hydrotalcite catalysts, where the metal is distributed within the hydrotalcite.

As stated above diethyl ether (DEE) and ethylene are made in the reaction mixture by ethanol dehydration in the presence of an acid. Surprisingly and unexpectedly, it has now been found that a magnesium catalyst inhibits the DEE and ethylene formation. For example, selectivity to DEE of less than 10%, and selectivity of ethylene less than 5%. In one embodiment, surprisingly and unexpectedly, it has also been found that a palladium catalyst could also inhibit the DEE formation. Specifically, the inventors found that the catalyst composition enhance the selectivity of butanol by suppressing the formation of DEE and ethylene.

Water is a byproduct when converting ethanol to butanol. Since water is more polar than ethanol, it is believed that water might compete with ethanol on the polar surface of the catalyst. The inventors have found that the surface polarity of the catalysts may be modified by introducing an organic metal precursor to the surface of the support to minimize the water/ethanol competition. The organic metal precursor may include pyridine, ammonium hydroxide tetramethylammonium hydroxide, tetrabutylammonium hydroxide, methyl amine, imidazole, and other suitable support modifiers. The organic metal precursors may be support modifiers that may adjust the chemical or physical properties of the support material such as the acidity or basicity of the support material. For purposes of the present invention, the support material is hydrotalcite. As such, the amount and residence time of ethanol on the surface of the catalyst maybe increased and thereby promoting the carbon-carbon capillary condensation.

The catalyst may further comprise other additives, examples of which may include: molding assistants for enhancing moldability; reinforcements for enhancing the strength of the catalyst; pore-forming or pore modification agents for formation of appropriate pores in the catalyst, and binders. Examples of these other additives include stearic acid, graphite, starch, cellulose, silica, alumina, glass fibers, silicon carbide, and silicon nitride. Preferably, these additives do not have detrimental effects on the catalytic performances, e.g., conversion and/or activity. These various additives may be added in such an amount that the physical strength of the catalyst does not readily deteriorate to such an extent that it becomes impossible to use the catalyst practically as an industrial catalyst.

In some embodiments the catalyst composition comprises a pore modification agent, such as oxalic acid. A preferred type of pore modification agent is thermally stable and has a substantial vapor pressure at a temperature below 300° C., e.g., below 250° C. In one embodiment, the pore modification agent has a vapor pressure of at least 0.1 kPa, e.g., at least 0.5 kPa, at a temperature between 150° C. and 250° C., e.g., between 150° C. and 200° C.

The pore modification agent has a relatively high melting point, e.g., greater than 60° C., e.g., greater than 75° C., to prevent melting during the compression of the catalyst into a slug, tablet, or pellet. Preferably, the pore modification agent comprises a relatively pure material rather than a mixture. As such, lower melting components will not liquefy under compression during formation of slugs or tablets. For example, where the pore modification agent is a fatty acid, lower melting components of the fatty acid mixtures may be removed as liquids by pressing. If this phenomenon occurs during slug or tablet compression, the flow of liquid may disturb the pore structure and produce an undesirable distribution of pore volume as a function of pore diameter on the catalyst composition. In other embodiments, the pore modification agents have a significant vapor pressure at temperatures below their melting points, so that they can be removed by sublimination into a carrier gas.

### Catalyst Preparation

Unlike previously disclosed hydrotalcites-metal complexes where the metals are mixed homogeneously to form the hydrotalcites, the process for preparing the catalyst of the present invention involves coating one or more metals on the surface of the hydrotalcites. The catalysts of the present invention may be synthesized by the following methods.

In a first aspect, a metal precursor, such as a metal oxide, and hydrotalcite are grinded and mixed together until the mixture appears to be homogenous and solid, generally about five minutes. The mixture may be grinded and mixed using methods that is well known by person skilled in the arts, for example, via mortar and pestle, or a mill. In another embodiment, the metal oxide and the hydrotalcite may be crushed prior to mixing. The homogeneous solid mixture is calcined. The initial temperature may range from 10° C. to 150° C., e.g., 30° C. to 120° C., or 50° C. to 90° C. The temperature ramping rate may be from 1° C. to 5° C. per minute. The final temperature may vary depending on the catalyst composition and generally ranges from 300° C. to 900° C., e.g., from 450° C. to 800° C., or from 500° C. to 700° C. The holding time is between 1 hour and 10 hours, e.g., between 2 hours and 8 hours, or between 4 hours and 6 hours. Depending on the metal used, other temperature profiles may be suitable. The calcination of the mixture may be done in an inert atmosphere, air or an oxygen-containing gas at the desired temperatures. Steam, a hydrocarbon or other gases or vapors may be added to the atmosphere during the calcination step or post-calcination to cause desired effects on physical and chemical surface properties as well as textural properties such as increase macroporosity.

As an example, the temperature profile may start at 60° C., increase at a rate of 5° C. per minute until the temperature reaches 600° C., and hold at 600° C. for 5 hours, and cooling to room temperature. For transition metals, the calcination temperature may be lower, such as 300° C.

The calcined composition is pressed, under force, for a period of time to form pellets. For example, composition may be pressed at least 10,000 N, e.g., at least 15,000 N, at least 20,000 N, or at least 25,000 N. In terms of ranges, the composition may be pressed between 10,000 N to 500,000 N, e.g., between 15,000 N to 400,000 N, or from between 20,000 N to 200,000 N. In one embodiment, the calcined composition is pressed under force for at least 5 minutes, e.g., at least 20 minutes, at least 40 minutes, or at least 60 minutes. In another embodiment, the calcined composition may be pressed under an increased pressure profile. For example, the calcined composition may be pressed under a certain pressure for a pre-set period of time and the pressure may increase gradually for another pre-set period of time. For example, the calcined composition may be pressed under 100,000 N for 1 minute, and then the pressure may increase by an additional 100,000 N for each minute until it reaches a pre-determined pressure.

The pellets are then lightly crushed to suitable particle sizes.

In a second aspect, a metal precursor, such as a metal nitrate, may be added to a solution of ammonium oxalate and the mixture may be heated until the solution is clear. Then, an amount of acetone and/or water may be added to the clear solution. The resulting solution may be added dropwise to hydrotalcite. The metal-coated hydrotalcite may be dried and calcined using a temperature profile similar to method of the previous aspect.

In another embodiment, a multi-layered metal-coated hydrotalcite may be prepared by adding a second or a third metal layer to the metal-coated hydrotalcite. For example, the metal-coated hydrotalcite may be cooled after calcinations and a second metal nitrate or metal oxide may be coated thereon. The twice-coated hydrotalcite may be dried and calcined before a third metal layer is added.

In one embodiment, any suitable metal precursors may be used to make the catalyst composition. The metal precursors may also be used for the secondary metals. The metal precursors may be selected from the group consisting of metal oxides, metal nitrates, metal acetate, and metal oxalate. Non-limiting examples of suitable metal precursors include metal oxides, metal hydroxides (including hydrated oxides), metal salts of inorganic and organic acids such as, e.g., nitrates, nitrites, sulfates, halides (e.g., fluorides, chlorides, bromides and iodides), carbonates, phosphates, azides, borates (including fluoroborates, pyrazolylborates, etc.), sulfonates, carboxylates (such as, e.g., formates, acetates, propionates, oxalates and citrates), substituted carboxylates (including halogenocarboxylates such as, e.g., trifluoroacetates, hydroxycarboxylates, aminocarboxylates, etc.), metal acetylacetonate, and salts and acids wherein the metal is part of an anion (such as, e.g., hexachloroplatinates, tetrachloroaurate, tungstates and the corresponding acids).

Further non-limiting examples of suitable metal precursors for the processes of the present invention include alkoxides, complex compounds (e.g., complex salts) of metals such as, e.g., beta-diketonates (e.g., acetylacetonates), complexes with amines, N-heterocyclic compounds (e.g., pyrrole, aziridine, indole, piperidine, morpholine, pyridine, imidazole, piperazine, triazoles, and substituted derivatives thereof), aminoalcohols (e.g., ethanolamine, etc.), amino acids (e.g., glycine, etc.), amides (e.g., formamides, acetamides, etc.), and nitriles (e.g., acetonitrile, etc.). Non-limiting examples of preferred metal precursors include nitrates and oxides.

Non-limiting examples of specific metal precursors for use in the processes of the present invention include germanium oxide, germanium butoxide, germanium glycolate, germanium chloride, germanium acetate, germanium hydroxide, germanium methoxide, germanium nitride, and bis(2-carboxyethyl germanium sesquioxide); tin(II) oxide, tin oxalate, tin acetate, tin chloride, and tin nitrate; palladium bromide, palladium chloride, palladium iodide, palladium nitrate, palladium nitrate hydrate, tetraamine palladium nitrate, palladium oxide, palladium oxide hydrate, and palladium sulfate; magnesium nitrate hexahydrate, magnesium nitrate, hydrated magnesium nitrate, magnesium chloride, hydrated magnesium chloride, magnesium chloride hexahydrate, magnesium acetate tetrahydrate, magnesium acetylacetonate dihydrate, magnesium carbonate hydroxide pentahydrate, magnesium perchlorate, magnesium perchlorate hexahydrate, magnesium sulfate, magnesium sulfate heptahydrate, and magnesium sulfate monohydrate; copper oxide, copper hydroxide, copper nitrate, copper sulfate, copper chloride, copper formate, copper acetate, copper neodecanoate, copper ethylhexanoate, copper methacrylate, copper trifluoroacetate, copper acetoacetate and copper hexafluoroacetylacetonate; lithium nitrate, lithium acetate, lithium acetate dehydrate, and lithium phosphate; potassium nitrate, potassium acetate, potassium sulfate, and potassium sulfite; cesium nitrate, cesium chloride, cesium hydroxide, cesium carbonate, cesium oxalate, cesium perchlorate, cesium propionate, and cesium formate; aluminum alkoxide, aluminum nitrate, aluminum hydroxide, aluminum oxide, aluminum acetate, aluminum sulfate, aluminum chloride, and aluminum bromide. The above compounds may be employed as such or optionally in the form of solvates and the like such as, e.g., as hydrates. Examples of specific metal precursors that may be used in the present invention include germanium oxide, bis(2-carboxyethyl germanium sesquioxide), tin(II) oxide, tin oxalate, palladium nitrate hydrate, magnesium nitrate hydrate, copper nitrate hydrate, aluminum nitrate hydrate, and lithium nitrate.

The use of mixtures of different compounds, e.g., different salts, of the same metal and/or the use of mixtures of compounds of different metals and/or of mixed metal precursors (e.g., mixed salts and/or mixed oxides) is also contemplated by the present invention. Accordingly, the term “metal precursor” as used herein includes both a single metal precursor and any mixture of two or more metal precursors. In a preferred embodiment, the catalyst composition is made using hydrotalcite and a first metal precursor, and optionally a second metal precursor and a third metal precursor.

### Production of Butanol

Suitable reactions and/or separation scheme may be employed to form a crude product stream comprising butanol using the catalysts. For example, in some embodiments, the crude product stream is formed by contacting a low molecular weight alcohol, e.g., ethanol, with the catalysts to form the crude higher alcohol product stream, i.e., a stream with butanol. Preferably, the catalyst is a metal-coated hydrotalcite. In a preferred embodiment, the crude product stream is the reaction product of the condensation reaction of ethanol, which is conducted over a metal-coated hydrotalcite. In one embodiment, the crude product stream is the product of a vapor phase reaction.

In some embodiments, the condensation reaction may achieve favorable conversion of ethanol and favorable selectivity and productivity to butanol. For purposes of the present invention, the term “conversion” refers to the amount of ethanol in the feed that is converted to a compound other than ethanol. Conversion is expressed as a percentage based on ethanol in the feed. The conversion of ethanol may be at least 28%, e.g., at least 30%, at least 40%, or at least 60%.

The feedstream may be a gaseous stream comprising ethanol. Preferably, the gaseous stream comprise more than 5 vol. % ethanol, e.g., more than 10 vol. % or more than 20 vol. %. The feedstream may also comprise other molecules such as pyridine, NH₃, and alkyl amine. Inert gases may be in the gaseous stream and thus may include nitrogen, helium, argon, and methane. Preferably, no hydrogen is introduced with the gaseous stream, and thus the gaseous stream is substantially free of hydrogen. Without being bound by theory the hydrogen needed for the intermediate reactions may be produced in situ.

Selectivity, as it refers to the formation of butanol, is expressed as the ratio of the amount of carbon in the desired product(s) and the amount of carbon in the total products. Preferably, the selectivity to butanol is at least 30%, e.g., at least 40%, or at least 60%. In some embodiments, the catalyst selectivity to C₄₊ alcohols, e.g., n-butanol, isobutanol, 2-butanol, tert-butanol, 1-hexanol, 2-ethylbutanol, or 2-ethylhexanol, is at least 30%, e.g., at least 50%, at least 60%, or at least 80%.

Preferred embodiments of the inventive process demonstrate a low selectivity to undesirable products, such as DEE and ethylene. The selectivity to these undesirable products preferably is less than 20%, e.g., less than 5% or less than 1%. More preferably, these undesirable products are not detectable.

The ethanol may be fed to the reactor as a liquid stream or a vapor stream. Preferably, the ethanol is fed as a vapor stream. The reactor may be any suitable reactor or combination of reactors. Preferably, the reactor comprises a fixed bed reactor or a series of fixed bed reactors. In one embodiment, the reactor is a gas flow catalytic reactor or a series of gas flow catalytic reactors. Of course, other reactors such as a continuous stirred tank reactor or a fluidized bed reactor, may be employed. In one embodiment, the vapor ethanol stream is substantially free of hydrogen, e.g., less than 1 wt. % hydrogen, less than 0.1 wt. %, or less than 0.01 wt. %.

The condensation reaction may be conducted at a temperature of at least 250° C., e.g., at least 300° C., or at least 350° C. In terms of ranges, the reaction temperature may range from 200° C. to 500° C., e.g., from 250° C. to 400° C., or from 250° C. to 350° C. Residence time in the reactor may range from 0.01 to 100 hours, e.g., from 1 to 80 hours, or from 5 to 80 hours. Reaction pressure is not particularly limited, and the reaction is typically performed near atmospheric pressure. In one embodiment, the reaction may be conducted at a pressure ranging from 0.1 kPa to 9,000 kPa, e.g., from 20 kPa to 5,000 kPa, or from 90 to 3500 kPa. The ethanol conversion may vary depending upon the reaction temperature and/or pressure.

In one embodiment, the reaction is conducted at a gas hourly space velocity (“GHSV”) greater than 600 hr⁻¹, e.g., greater than 1000 hr⁻¹ or greater than 2000 hr⁻¹. The GHSV may range from 600 hr⁻¹ to 10000 hr⁻¹, e.g., from 1000 hr⁻¹ to 8000 hr⁻¹ or from 1500 hr⁻¹ to 7500 hr⁻¹.

An inert or reactive gas may be supplied to the reactant stream. Examples of inert gases include, but are not limited to, nitrogen, helium, argon, and methane. Examples of reactive gases or vapors include, but are not limited to, oxygen, carbon oxides, sulfur oxides, and alkyl halides. When reactive gases such as oxygen are added to the reactor, these gases, in some embodiments, may be added in stages throughout the catalyst bed at desired levels as well as feeding with the other feed components at the beginning of the reactors. The addition of these additional components may improve reaction efficiencies.

In one embodiment, the unreacted components such as the ethanol as well as the inert or reactive gases that remain are recycled to the reactor after sufficient separation from the desired product.

### EXAMPLES

### Preparation by Pressing Powder to Form Catalyst

### Example 1

### Tin Coated Hydrotalcite (Sn-HT)

0.355 g of SnO and 10 g of hydrotalcite were mixed together via mortar and pestle (3 wt. % Sn) for approximately 5 minutes until the black and white powders appeared homogenous. The solid mixture was then calcined using the following temperature program: start at 60° C., ramp to 600° C. at 5° C./min, hold at 600° C. for 5 hours, followed by cooling to room temperature. The tin coated hydrotalcite powder was then pressed at 180,000 N for 1 hour to form pellets, followed by lightly crushed the above pellets to a particle size of 0.85 mm and 1.18 mm for further use.

### Example 2

### Germanium Coated Hydrotalcite (Ge-HT)

0.45 g of GeO₂ and 10 g of hydrotalcite were mixed together via mortar and pestle (3 wt. % Ge) for approximately 5 minutes until the black and white powders appeared homogenous. The solid mixture was then calcined using the following temperature program: start at 60° C., ramp to 600° C. at 5° C./min, hold at 600° C. for 5 hours, followed by cooling to room temperature. The germanium coated hydrotalcite powder was then pressed at 180,000 N for 1 hour to form pellets, followed by lightly crushed the above pellets to a particle size of 0.85 mm and 1.18 mm for further use.

### Preparation Through Impregnation, Drying and Calcination

### Example 3

### Tin Coated Hydrotalcite (Sn-HT)

0.56 g of ammonium oxalate was dissolved in 10 g of distilled H₂O, followed by adding 0.54 g of tin oxalate to the solution. The resulted mixture was then heated at 60° C. until the solution turns clear, and then 1 g of acetone was added to the clear solution. The resulted clear solution was impregnated to 10 g of hydrotalcite by stepwise incipient wetness using a rotating dryer. The obtained samples were dried in the oven at 120° C. for 5 hours, followed by calcination using the following temperature program: start at 60° C., ramp to 600° C. at 2° C./min, hold at 600° C. for 5 hours, followed by cooling to room temperature.

### Example 4

### Germanium Coated Hydrotalcite (Ge-HT)

0.36 g of bis(2-carboxyethyl germanium (IV) sesquioxide) was added to 25 g of distilled water and heated at about 65° C. until the solution was clear, followed by adding 1 g of acetone. The resulted clear solution was impregnated to 5 g of hydrotalcite by stepwise incipient wetness using a rotating dryer. The obtained samples were dried and calcined under the conditions described in Example 3.

### Example 5

### Palladium Coated Hydrotalcite (Pd-HT)

Hydrotalcite powder was pressed at 180,000 N for 1 hour to form pellets. The pellets were then lightly crushed to a particle size of 0.85 mm and 1.18 mm. 5 g of the lightly crushed hydrotalcite pellets were measured and placed in a round bottom reactor. 0.39 g of Pd(NO₃)₂.2H₂O was dissolved in the mixture of 5 g of water and 5 g of acetone. The resulted solution was impregnated to 5 g of the hydrotalcite by stepwise incipient wetness using a rotating dryer. The obtained samples were dried and calcined under the conditions described in Example 3.

### Example 6

### Gallium Coated Hydrotalcite (Ga-HT)

10 g of the lightly crushed hydrotalcite pellets, as prepared in Example 5, were measured and placed in a round bottom reactor. 1.13 g of Ga(NO₃)₃ was dissolved in 10 g of water. The resulted solution was impregnated to 10 g of the hydrotalcite by stepwise incipient wetness using a rotating dryer. The obtained samples were dried and calcined under the conditions described in Example 3.

### Example 7

### Copper-Magnesium Coated Hydrotalcite (Cu—Mg-HT)

10 g of the lightly crushed hydrotalcite pellets, as prepared in Example 5, was measured and placed in a round bottom reactor. The 1^{st} layer coating of magnesium to hydrotalcite was prepared by dissolving 0.45 g of Mg(NO₃)₂.2H₂O in a mixture of 5 g of water and 5 g of acetone. The resulted solution was impregnated to 5 g of the hydrotalcite by stepwise incipient wetness using the rotating dryer. The obtained samples were dried and calcined under the conditions described in Example 3. A second layer coating of copper was made the same way as the first layer, i.e., by dissolving 0.545 g of Cu(NO₃)₂.2H₂O (Mole ratio of Cu/Mg=1:1) was dissolved in the mixture of 5 g of water and 5 g of acetone. The resulted solution was impregnated to the above magnesium coated hydrotalcite by stepwise incipient wetness using the rotating dryer. The obtained samples were dried in the oven at 120° C. for 5 hours, followed by calcination using the following temperature program: start at 60° C., ramp to 300° C. at 2° C./min, hold at 300° C. for 5 hours, followed by cooling to room temperature.

### Example 8

### 3 wt. % Palladium Lithium Hydrotalcite

The catalyst was synthesized using sequential impregnation method with lithium on the inner layer and palladium on the outer layer. 10 g of the prepared hydrotalcite pellets, as prepared in Example 5, was measured and placed in a round bottom reactor. The lithium layer on the hydrotalcite was prepared by dissolving 0.24 g of lithium nitrate in 5 g of water and 5 g of acetone, followed by impregnating to the hydrotalcite by stepwise incipient wetness using the rotating dryer. The lithium coated hydrotalcite was then dried and calcined under the conditions described in Example 3. The palladium layer was prepared by dissolving 0.773 g of palladium (II) nitrate hydrate in 5 g of water and 5 g of acetone, followed by impregnating to the lithium coated hydrotalcite by stepwise incipient wetness using the rotating dryer. The palladium-lithium coated hydrotalcite was dried and calcined under similar conditions as above.

### Example 9

### Palladium-Magnesium Coated Hydrotalcite (Pd—Mg-HT)

10 g of the lightly crushed hydrotalcite pellets, as prepared in Example 5, was measured and placed in a round bottom reactor. The 1^{st} layer coating of magnesium to hydrotalcite was prepared by dissolving 0.270 g of Mg(NO₃)₂.2H₂O in the mixture of 5 g of water and 5 g of acetone. The resulted solution was impregnated to 5 g of the hydrotalcite by stepwise incipient wetness using the rotating dryer. The obtained samples were dried and calcined under the conditions described in Example 3. A second layer of palladium was made the same way as the first layer, i.e., by dissolving 0.39 g of Pd(NO₃)₂.2H₂O (mole ratio of Pd/Mg=1:1) in a mixture of 5 g of water and 5 g of acetone. The resulted solution was impregnated to the above magnesium coated hydrotalcite by stepwise incipient wetness using the rotating dryer. The obtained samples were dried in the oven at 120° C. for 5 hours, followed by calcination using the following temperature program: start at 60° C., ramp to 300° C. at 2° C./min, hold at 300° C. for 5 hours, followed by cooling to room temperature.

### Example 10

### Copper-Magnesium/Aluminum Coated Hydrotalcite (Cu—Mg/Al-HT)

10 g of the lightly crushed hydrotalcite pellets, as prepared in Example 5, was measured and placed in a round bottom reactor. The 1^{st} layer of magnesium/aluminum to hydrotalcite was prepared by dissolving 0.45 g of Mg(NO₃)₂.2H₂O and 0.305 g of Al(NO₃)₃.9H₂O (Mg/Al mole ratio=3:1) in 5 g of water and 5 g of acetone. The resulted solution was slowly impregnated to 5 g of the hydrotalcite in a reactor by stepwise incipient wetness using the rotating dryer. The obtained samples were dried and calcined under the conditions described in Example 3. A second layer of copper was made the same way as the first layer, i.e., by dissolving 0.545 g of Cu(NO₃)₂.2H₂O (mole ratio of Cu/Mg=1:1) in 5 g of H₂O and 5 g of acetone. The resulted solution was slowly added to the above Mg/Al coated HT in the reactor by stepwise incipient wetness using the rotating dryer. The obtained samples were dried and calcined under similar conditions.

### Example 11

The above-prepared catalysts were evaluated. Hydrotalcite without any metal coating was also evaluated under the same testing conditions and served as control. A fixed bed gas flow catalytic reactor was used as a reactor. 3 ml of above prepared catalysts was filled in a stainless steel tube reactor with a diameter of 0.95 cm. As a pretreatment, hydrogen reduction was conducted for 1 hour under a carrier gas atmosphere (10% H₂/N₂ base; flow rate 125 ml/min) at 400° C. After the pretreatment, the testing was conducted at a temperature between 250° C. and 325° C. and pressure between 1 kPa and 5,100 kPa, nitrogen flow rate was at 125 sccm and ethanol flow rate at 0.2 ml/min. The reaction duration ranges from 5˜80 hrs.

The ethanol conversion, butanol product selectivity, butanol yield, and C₄₊ alcohol selectivity for catalyst metal-coated hydrotalcite and hydrotalcite as reference are shown in Tables 1 to 9.

TABLE 1
Uncoated HT vs. Sn coated HT
Testing condition: 400° C. and 1,700 kPa

| | Ethanol | Butanol | Yield | C₄₊ Alcohols |
|---|---|---|---|---|
| Catalysts | Conversion (%) | Selectivity (%) | (%) | Selectivity (%) |
| Uncoated HT | 45 | 42 | 19 | 54 |
| Example 1 | 49 | 52 | 25 | 75 |
| Example 3 | 57 | 50 | 28 | 76 |

Example 1 tin-coated hydrotalcite was made using method 1 and example 3 tin-coated hydrotalcite was made using method 2. As shown in Table 1, under the same testing condition, both tin-coated hydrotalcite have better ethanol conversion, butanol selective, butanol yield, and C₄₊ alcohols selectivity than the uncoated hydrotalcite. Tin-coated hydrotalcite made using the impregnation method surprisingly provide better ethanol conversion and slightly better yield and C₄₊ alcohols selectivity than the tin-coated hydrotalcite made using the mixing method.

TABLE 2
Uncoated HT vs. Pd—Li coated HT
Testing condition: 290° C. and 3,400 kPa

| | Ethanol | | | C₄₊ Alcohols | DEE | Ethylene |
|---|---|---|---|---|---|---|
| | Conversion | Butanol | | Selectivity | Selectivity | Selectivity |
| Catalysts | (%) | Selectivity (%) | Yield (%) | (%) | (%) | (%) |
| Uncoated | 13 | 27 | 3.5 | 33 | 63 | 2 |
| HT | | | | | | |
| Example 8 | 50 | 49 | 24 | 65 | 0 | 17 |

As shown in Table 2, under the same condition, palladium-lithium coated hydrotalcite has better ethanol conversion, butanol selectivity, butanol yield, and C₄₊ alcohols selectivity than the metal free hydrotalcite. Furthermore, the metal coated hydrotalcite catalysts significantly reduce the DEE selectivity to zero. Selectivity for butanol busing the metal coated hydrotalcite is greater than the ethylene selectivity.

TABLE 3
Uncoated HT vs. Cu—Mg coated HT, Pd—Mg
coated HT and Cu—Mg/Al coated HT
Testing condition: 400° C. and 3,400 kPa

| | Ethanol | Butanol | Yield | C₄₊ Alcohols |
|---|---|---|---|---|
| Catalysts | Conversion (%) | Selectivity (%) | (%) | Selectivity (%) |
| Uncoated HT | 22 | 26 | 6 | 37 |
| Example 7 | 57 | 57 | 32 | 77 |
| Example 9 | 51 | 56 | 29 | 72 |
| Example 10 | 50 | 58 | 29 | 75 |

As shown in Table 3, under the same testing condition, the metal coated hydrotalcites out perform the uncoated hydrotalcite on ethanol conversion, butanol selective, butanol yield, and C₄₊ alcohols selectivity.

TABLE 4
Uncoated HT vs. Ge coated HT and Cu—Mg/Al coated HT
Testing condition: 360° C. and 3,400 kPa

| | Ethanol | Butanol | Yield | C₄₊ Alcohols |
|---|---|---|---|---|
| Catalysts | Conversion (%) | Selectivity (%) | (%) | Selectivity (%) |
| Uncoated HT | 40 | 54 | 22 | 67 |
| Example 2 | 35 | 63 | 22 | 72 |
| Example 4 | 41 | 57 | 29 | 70 |
| Example 10 | 39 | 58 | 23 | 75 |

As shown in Table 4, under the same testing condition, the metal coated hydrotalcites have similar ethanol conversion, butanol selective, butanol yield, and C₄₊ alcohols selectivity to the uncoated hydrotalcite. The higher selectivity to butanol in Examples 2, 3 and 4 may also reduce the purification requirements when recovering butanol.

TABLE 5
Uncoated HT vs. Pd coated HT

| | | Ethanol | Butanol | | C₄₊ Alcohols |
|---|---|---|---|---|---|
| | Testing | Conver- | Selectiv- | Yield | Selectivity |
| Catalysts | Conditions | sion (%) | ity (%) | (%) | (%) |
| Uncoated HT | 290° C. | 13 | 28 | 4 | 33 |
| | 1,700 kPa | | | | |
| Uncoated HT | 290° C. | 13 | 27 | 3 | 33 |
| | 3,400 kPa | | | | |
| Example 5 | 290° C. | 28 | 70 | 20 | 82 |
| | 1,700 kPa | | | | |
| Example 5 | 290° C. | 54 | 63 | 34 | 84 |
| | 3,400 kPa | | | | |

As shown in Table 5, samples uncoated HT and palladium coated hydrotalcite were tested under different pressures. It appears that increase in pressure has no effect in uncoated hydrotalcite. In comparison, the increase in pressure increased the ethanol conversion of palladium hydrotalcite from 28% to 54% and increased the yield from 20% to 34%. In addition, when comparing uncoated hydrotalcite and palladium coated hydrotalcite, under the same testing condition, palladium coated hydrotalcite has higher ethanol conversion, butanol selective, butanol yield, and C₄₊ alcohols selectivity than uncoated hydrotalcite.

TABLE 6
Pressure and Temperature Impact on Pd coated HT (Example 5)

| | | Ethanol | Butanol | | C₄₊ Alcohols |
|---|---|---|---|---|---|
| Temper- | | Conversion | Selectivity | Yield | Selectivity |
| ature | Pressure | (%) | (%) | (%) | (%) |
| 250° C. | 0 kPa | 7 | 13 | 1 | 14 |
| | 1,700 kPa | 16 | 47 | 8 | 56 |
| | 3,400 kPa | 67 | 67 | 28 | 86 |
| 290° C. | 1,700 kPa | 28 | 70 | 20 | 82 |
| | 3,400 kPa | 54 | 63 | 34 | 84 |
| | 5,100 kPa | 61 | 57 | 35 | 84 |
| 325° C. | 1,000 kPa | 34 | 15 | 5 | 16 |
| | 5,100 kPa | 45 | 60 | 27 | 68 |

Table 6 illustrates the pressure and temperature impact on palladium coated hydrotalcite. Samples of palladium coated hydrotalcite were tested under various temperature and pressure. At 250° C., as pressure increase from 0 kPa to 3,400 kPa, the ethanol conversion, butanol selectivity, butanol yield, and C₄₊ alcohols selectivity all increase as pressure increases. Surprisingly and unexpectedly, at 290° C., while ethanol conversion and yield increased, butanol selectivity decreased and C₄₊ alcohols selectivity remained steady. At 325° C., increase of pressure appears to also increase ethanol conversion, butanol selectivity, butanol yield and C₄₊ alcohols selectivity.

TABLE 7
Uncoated HT vs. Ga-HT

| | | Ethanol | Butanol | | C₄₊ Alcohols |
|---|---|---|---|---|---|
| | Testing | Conversion | Selectivity | Yield | Selectivity |
| Catalysts | Conditions | (%) | (%) | (%) | (%) |
| Uncoated | 290° C. | 13 | 28 | 4 | 33 |
| HT | 1,700 kPa | | | | |
| Example 6 | 270° C. | 28 | 70 | 13 | 90.3 |
| | 1,700 kPa | | | | |

As shown in Table 7, the reaction for the uncoated hydrotalcite was conducted at 290° C. and the reaction for the gallium coated hydrotalcite was conducted at 270° C. Even at a lower temperature, Gallium coated hydrotalcite has higher ethanol conversion, butanol selective, butanol yield and C₄₊ alcohols selectivity than uncoated hydrotalcite.

TABLE 8
Uncoated HT vs. Pd—Mg coated HT
Testing condition: 250° C. and 3,400 kPa

| | Ethanol | Butanol | Yield | C₄₊ Alcohols |
|---|---|---|---|---|
| Catalysts | Conversion (%) | Selectivity (%) | (%) | Selectivity (%) |
| HT | 0.4 | 36 | 0.1 | 61 |
| Example 9 | 39 | 53 | 21 | 73 |

As shown in Table 8, under the same testing condition, palladium-magnesium coated hydrotalcite has better ethanol conversion and higher butanol selective, butanol yield, and C₄₊ alcohols selectivity than uncoated hydrotalcite.

Therefore, under every testing condition, the metal-coated hydrotalcite catalysts have similar or better ethanol conversion, butanol selectivity, butanol yield, and C₄₊ alcohols selectivity than uncoated hydrotalcite. It is postulated that the coating of metal(s) on hydrotalcite provides additional functionality to the catalyst composition and beneficially drives the reaction to higher selectivity and conversion.

### Comparative Example

U.S. Pat. No. 7,700,811 discloses hydrotalcite/metal carbonate composition. According to Example 1 of 7,700,811, the catalyst composition was made by combining aluminum nitrate, magnesium nitrate, and copper nitrate. Therefore, unlike the claimed catalyst, the catalyst of 7,700,811 comprises a homogeneous distribution of copper, aluminum, and magnesium. Table 7 provides a comparison between the copper-magnesium-aluminum catalyst of 7,700,811 (Comp. A) and the hydrotalcite-magnesium/aluminum-copper catalyst of the present invention (Example 10).

TABLE 9
Homogeneous Copper-Magnesium-Aluminum catalyst
vs. Cu—Mg/Al coated Hydrotalcite

| | Ethanol | Butanol | |
|---|---|---|---|
| Catalysts | Conversion (%) | Selectivity (%) | Yield (%) |
| Comp. A | 30 | 52.7 | 15.9 |
| Example 10 | 68 | 58 | 39 |

As shown in Table 9, the catalyst according to the present invention outperformed the catalyst disclosed in 7,700,811 in ethanol conversion, butanol selectivity and yield. It is postulated that the metal coating on the surface of hydrotalcite beneficially drives the Guerbet reaction favorably for both ethanol conversion and selectivity to butanol as compared to homogeneous co-precipitation of metal-hydrotalcite catalyst, where the metal is distributed within the hydrotalcite.

While the invention has been described in detail, modifications within the spirit and scope of the invention will be readily apparent to those of skill in the art. It should be understood that aspects of the invention and portions of various embodiments and various features recited above and/or in the appended claims may be combined or interchanged either in whole or in part. In the foregoing descriptions of the various embodiments, those embodiments which refer to another embodiment may be appropriately combined with other embodiments as will be appreciated by one of ordinary skill in the art. Furthermore, those of ordinary skill in the art will appreciate that the foregoing description is by way of example only, and is not intended to limit the invention.

We claim:

1. A process for producing a catalyst composition for converting alcohols to higher alcohols, the process comprising:
coating hydrotalcite with one or more metal precursors, wherein the one or more metal precursors comprise one or more metals selected from the group consisting of magnesium, aluminum, gallium, germanium, tin, lead, copper, and other transition metals to form a metal-containing hydrotalcite; and
calcining the metal-containing hydrotalcite to form the catalyst composition.

2. The process of claim 1, further comprising coating hydrotalcite with one or more secondary metal precursors, wherein the one or more metal secondary precursors comprise metals selected from the group consisting of lithium, sodium, potassium, rubidium, cesium, francium, magnesium, calcium, strontium, and barium.

3. The process of claim 1, wherein the metal is selected from the group consisting of magnesium, aluminum, gallium, germanium, tin, palladium, and copper.

4. The process of claim 1, wherein each of the one or more metal precursors are coated on a surface of the hydrotalcite.

5. The process of claim 1, wherein the one or more metal precursors are selected from the group consisting of metal oxides, metal nitrates, metal acetate, metal oxalate, and metal acetylacetonate.

6. The process of claim 1, wherein the coating step comprises mixing and grinding the one or more metal precursors with the hydrotalcite to form a metal-containing hydrotalcite.

7. The process of claim 6, further comprising the steps of pressing the metal-containing hydrotalcite under pressure to form the catalyst.

8. The process of claim 7, wherein the pressure is between 10,000 N and 500,000 N.

9. The process of claim 1, wherein the coating step comprises impregnating hydrotalcite with the one or more metal precursors.

10. The process of claim 1, wherein the calcining step is conducted at a first temperature between 10° C. and 150° C. and a second temperature between 300° C. and 900° C.

11. A catalyst composition for converting alcohols to higher alcohols produced according to the process of claim 1.

12. The catalyst composition of claim 11, wherein the catalyst comprises from 70 wt. % to 99.9 wt. % hydrotalcite and from 0.1 wt. % to 30 wt. % metal.

13. The catalyst composition of claim 11, wherein the catalyst is of the formula:
HT-M,
wherein HT is Mg₆Al₂CO₃(OH)₁₆.4(H₂O), and
wherein M is one or more metals selected from the group consisting of magnesium, aluminum, gallium, germanium, tin, lead, copper, and other transition metals.

14. The catalyst composition of claim 13, M is selected from the group consisting of gallium, germanium, tin, palladium, magnesium/copper, magnesium/palladium, and magnesium/aluminum/copper.

15. A process for producing higher alcohols, the process comprising the steps of:
feeding a gaseous stream comprising ethanol over a catalyst composition in a reactor to form butanol, wherein the catalyst composition comprises a hydrotalcite coated with one or more metals, wherein the one or more metals are selected from the group consisting of magnesium, aluminum, gallium, germanium, tin, lead, copper, and other transition metals.

16. The process of claim 15, wherein the one or more metals are selected from the group consisting of magnesium, aluminum, gallium, germanium, tin, palladium, and copper.

17. The process of claim 15, wherein ethanol conversion is at least 28%.

18. The process of claim 15, wherein butanol selectivity is at least 30%.

19. The process of claim 15, wherein the catalyst comprises from 70 wt. % to 99.9 wt. % hydrotalcite and from 0.01 wt. % to 30 wt. % metal.

20. The process of claim 15, wherein the catalyst is of the formula:
HT-M, wherein M is selected from the group consisting of gallium, germanium, tin, palladium, magnesium/copper, magnesium/palladium, and magnesium/aluminum/copper.

21. A catalyst composition for converting alcohols to higher alcohols, where the catalyst is of the formula:
HT-Mₐ-M′_{b}-M″_{c}-A_{d},
wherein HT is Mg₆Al₂CO₃(OH)₁₆.4(H₂O);
M is gallium, germanium, tin, palladium, or magnesium;
M′ is magnesium, aluminum, or copper; and
M″ is aluminum or copper, provided that M, M′, and M″ are not the same;
A is lithium, sodium, potassium, rubidium, cesium, or francium;
a is 0.001 to 1,
b is 0 to 2,
c is 0 to 2; and
d is 0 to 2.

22. The catalyst composition of claim 21, wherein M, M′, M″, A, or combinations thereof is coated on HT.
